(19) United States
(12) Patent Application Publication
(10) Pub. No.: US 2016/0022213 A1
(43) Pub. Date: Jan. 28, 2016

(54) CLIP ADAPTOR FOR AN ACTIVITY MONITOR DEVICE AND OTHER DEVICES

(71) Applicant: Salutron, Inc., Fremont, CA (US)

(72) Inventor: Yong Jin Lee, Palo Alto, CA (US)

(21) Appl. No.: 14/341,226

(22) Filed: Jul. 25, 2014

(51) Int. Cl. A61B 5/00; A61B 5/0408; A61B 5/11; A61B 5/0205

(52) U.S. Cl. CPC A61B 5/6838; A61B 5/6831; A61B 5/6826; A61B 5/6824; A61B 5/02055; A61B 5/0408; A61B 5/0059; A61B 5/1118; A61B 5/4866; A61B 5/01

(57) ABSTRACT

Described herein is an adaptor configured to be selectively mated with a device, such as an activity monitor, to enable the device to be clipped to a person's finger or clothes, instead of being strapped around a wrist. The adaptor includes a base, lever and hinge. At least one connector extending from the base and is configured to mate with a portion of the device to thereby selectively mate the adaptor with the device. The base includes an opening sized and positioned to enable a distal portion of a person's finger to extend through the opening and contact the device when the adaptor and device are mated and the device is clipped to a person's finger. This enables the persons' skin to be in contact with at least one sensor of the device, to thereby enable the device to perform functions that rely on the sensor(s) contacting a persons' skin.

### BACKGROUND

Activity monitors have become popular as a tool for promoting exercise and a healthy lifestyle. In addition to keeping time, an activity monitor can include one or more sensors that are used to measure various metrics, such as heart rate, heart rate variability, calories burned, steps taken, and/or distance walked and/or ran. Moreover, user-specific information such as age, gender, height and weight can be used to tailor the measurements to the user. Such monitors can be worn on the wrist or arm, for instance. Such a monitor can be worn during an intended workout period or as a general, all day, free living monitor, where the user may perform specific exercises at some times while going about their daily activities at other times, e.g., including sitting, standing and sleeping.

A typical activity monitor may be affixed to a wrist or arm using portions of a band that fold around a wrist or arm, and then affix to each other with a clasp, or the like. In other words, an activity monitor may have the form factor of a typical wrist watch, and indeed, may also provide time keeping capabilities so the wearer need not also wear a wrist watch.

Some people prefer not to wear anything on their wrist or arm while exercising, e.g., because they find it uncomfortable. Accordingly, rather than strapping their activity monitor on their wrist or arm, some people may hold their activity monitor in one hand while exercising, which is inconvenient and/or cumbersome. Additionally, where the user needs both of their hands to perform an exercise, they may end up putting down their activity monitor, during which period of time the activity monitor will not perform its intended functions. Other people may choose to place their activity monitor in a pocket, where it is not viewable and is often uncomfortable. Depending upon what features are provided by the activity monitor, certain features may not function unless the activity monitor is in contact with a user's skin, and thus, some features may not function when the activity monitor is placed in a pocket.

### SUMMARY

Certain embodiments described herein relate to an adaptor configured to be selectively mated with a device, such as an activity monitor, to enable the device to be clipped to a person's finger or clothes, instead of being strapped around a person's wrist. In accordance with an embodiment, the adaptor includes a base, a lever and a hinge. The hinge connects a first end of the lever to a first end of the base and biases a second end of the lever towards a second end of the base. At least one connector extends from the base and is configured to mate with a portion of the device to thereby selectively mate the adaptor with the device. The base includes an opening sized and positioned to enable a distal portion of a person's finger to extend through the opening and contact the device when the adaptor is mated to the device and the device is clipped to a person's finger. This enables the persons' skin to be in contact with one or more sensors of the device, to thereby enable the device to perform functions that rely on the sensor(s) contacting a persons' skin. Embodiments also relate to systems that include a device, such as an activity monitor device, a detachable band that enables the device to be strapped to a persons' wrist, and an adaptor that enables the device to be clipped to a person's finger or clothes when the band is detached from the device. Embodiments also relate to methods for use with the above described system, adaptor and device.

This summary is provided to introduce a selection of concepts in a simplified form that are further described below in the description. This summary is not intended to identify key features or essential features of the claimed subject matter, nor is it intended to be used to limit the scope of the claimed subject matter. Further and alternative embodiments, and the features, aspects, and advantages of the embodiments of invention will become more apparent from the detailed description set forth below, the drawings and the claims.

### BRIEF DESCRIPTION OF THE DRAWINGS

FIGS. 1A, 1B and 1C are respectively front, back and perspective views of an exemplary activity monitor device having a band attached to it that enables the device to be strapped to a person's wrist.

FIG. 2 depicts a high level block diagram of exemplary electrical components of the device of FIGS. 1A-1C.

FIGS. 3A, 3B and 3C respectively show front perspective, rear perspective and side views of the activity monitor device of FIGS. 1A-1C after the band shown in FIGS. 1A-1C has been detached from the device.

FIGS. 4A, 4B and 4C respectively show front perspective, rear perspective and side views of an adaptor, according to an embodiment of the present invention, that can be selectively mated with the activity monitor device of FIGS. 1A-1C after the band shown in FIGS. 1A-1C has been detached from the device.

FIG. 5 illustrates how the adapter of FIGS. 4A-4C can be mated with the activity monitor device of FIGS. 1A-1C.

FIGS. 6A and 6B respectively show side and perspective views of the adaptor of FIGS. 4A-4C mated with the activity monitor device of FIGS. 1A-1C.

FIG. 7 illustrates additional details of the connectors of the adaptor of FIGS. 4A-4C, and additional details of the slots of the device of FIGS. 1A-1C to which the adaptor is configured to mate.

FIG. 8 illustrates the device of FIGS. 1A-1C with the adapter of FIGS. 4A-4C mated therewith and clipped to a finger of a person's right hand, while a finger of the person's left hand is touching an outwardly facing ECG sensor of the device.

FIG. 9 is a high level flow diagram that is used to describe methods according to embodiments of the present invention.

### DETAILED DESCRIPTION

In the following detailed description, reference is made to the accompanying drawings that form a part hereof, and in which is shown by way of illustration specific illustrative embodiments. It is to be understood that other embodiments may be utilized and that mechanical and electrical changes may be made. The following detailed description is, therefore, not to be taken in a limiting sense. In the description that follows, like numerals or reference designators will be used to refer to like parts or elements throughout. In addition, the first digit of a reference number identifies the drawing in which the reference number first appears.

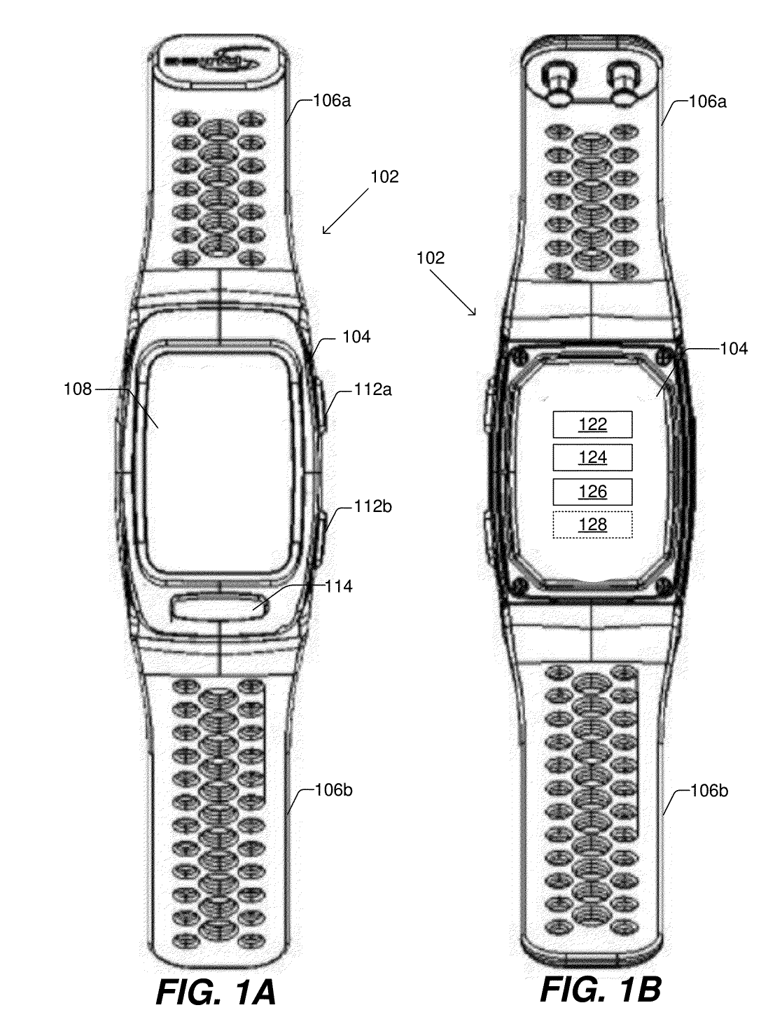

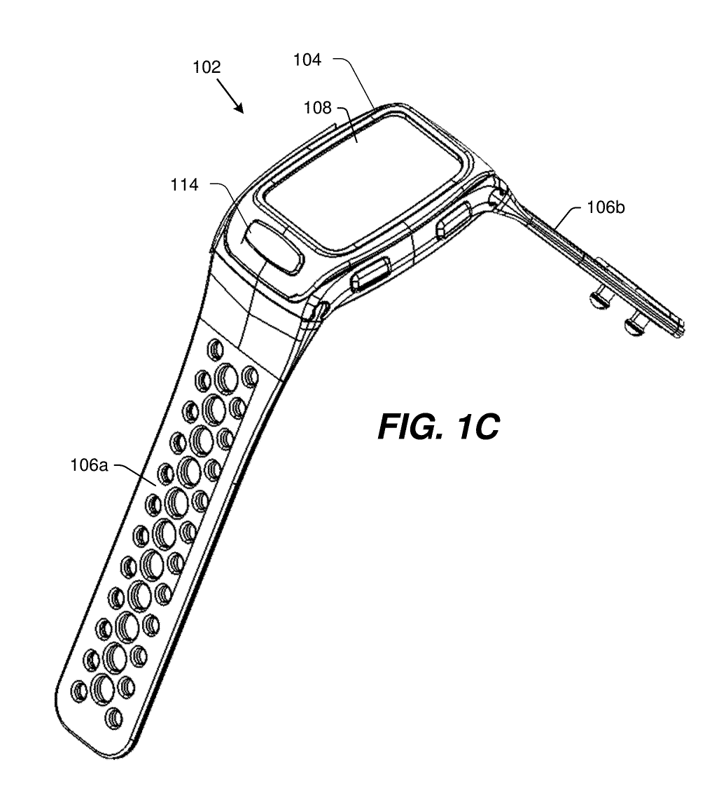

FIGS. 1A, 1B and 1C are respectively front, back and perspective views of a device 102 having a band attached to it that enables the device 102 to be strapped to a person's wrist. Depending upon the length of the band, the device 102 may alternatively be strapped to a user's forearm, bicep or ankle. The band includes first and second portions of the band 106a, 106b, which can collectively be referred to as the band 106. The device 102 is shown as including a housing 104, which can also be referred to as a case 104. The housing 104 is shown as including a digital display 108, which can also be referred to simply as a display.

The device 102 can be an activity monitor device, which can also be referred to simply as an activity monitor. In such an embodiment, the display 108 can be used to display activity and/or physiological metrics, such as, but not limited to, heart rate (HR), heart rate variability (HRV), calories burned, steps taken and distance walked and/or run. Additionally, the digital display 108 can be used to show the time, date, day of the week and/or the like. These are just examples of the types of information that may be displayed on the digital display 108, which are not intended to be all encompassing.

The housing 104 is also shown as including buttons 112a, 112b, which can individually be referred to as a button 112, and can collectively be referred to as the buttons 112. One of the buttons 112 can be a mode select button, while another one of the buttons 112 can be used to start and stop certain features. While the device 102 is shown as including two buttons 112, more or less than two buttons can be included. The buttons 112 can additionally or alternatively be used for other functions. The housing 104 is further shown as including a forward outward facing ECG electrode 114, which is discussed below, and which can also be referred to as an ECG sensor. This ECG electrode 114 can also function as an additional button.

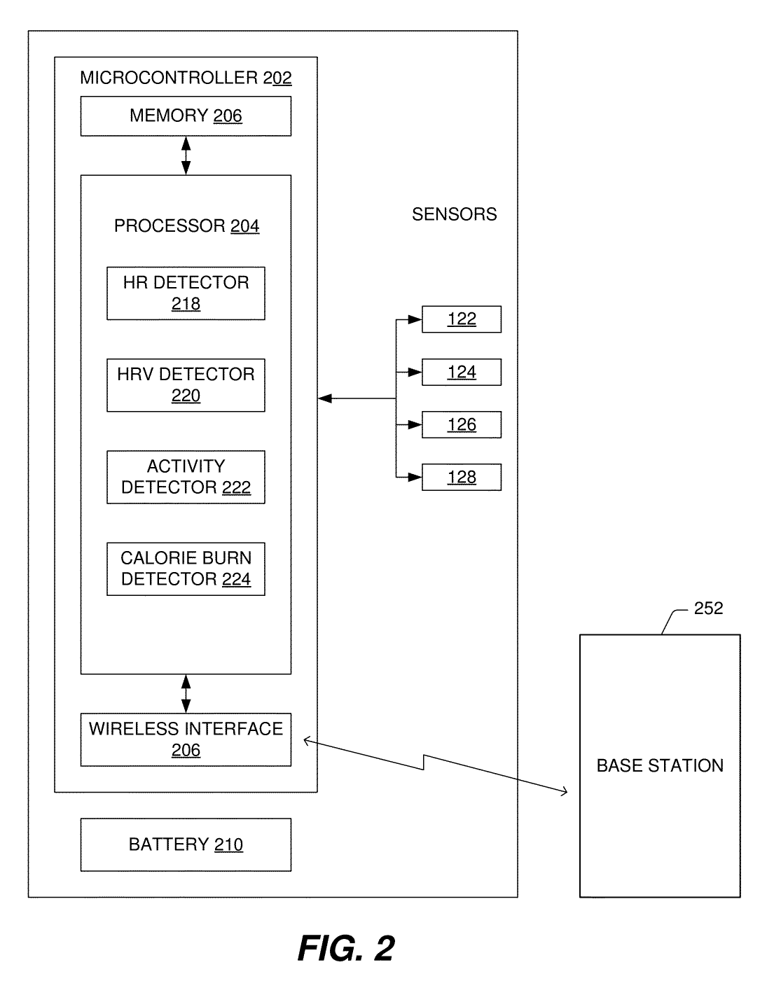

In certain embodiments, the device 102 can receive alerts from a base station (e.g., 252 in FIG. 2). For example, where the base station 252 is a mobile phone, the device 102 can receive alerts from the base station, which can be displayed to the user on the display 108. For a more specific example, if a mobile phone type of base station 252 is receiving an incoming phone call, then an incoming phone call alert can be displayed on the digital display 108 of the mobile device, which may or may not include the phone number and/or identity of the caller. Other types of alerts include, e.g., text message alerts, social media alerts, calendar alerts, medication reminders and exercise reminders, but are not limited thereto. The device 102 can inform the user of a new alert by vibrating and/or emitting an audible sound.

Referring to FIG. 1B, the backside of the housing 104 is shown as including an optical sensor 122, an electrocardiogram (ECG) sensor 124 and a skin temperature sensor 126. It is also possible that the device 102 includes less sensors than shown, more sensors than shown and/or alternative types of sensors. For example, the device 102 can also include one or more type of motion sensor 128, which is shown in dotted line because it is likely complete encased with the housing 104.

The optical sensor 122 can include both a light source and a light detector, in which case the optical sensor 122 can be used to detect HR and HRV. More specifically, the optical sensor 122 can operate as a photoplethysmography (PPG) sensor. When operating as a PPG sensor, the light source of the optical sensor 122 emits light that is reflected or backscattered by patient tissue, and reflected/backscattered light is received by the light detector of the optical sensor 122. In this manner, changes in reflected light intensity are detected by the light detector, which outputs a PPG signal indicative of the changes in detected light, which are indicative of changes in blood volume. The PPG signal output by the light detector can be filtered and amplified, and can be converted to a digital signal using an analog-to-digital converter (ADC), if the PPG signal is to be analyzed in the digital domain. Each cardiac cycle in the PPG signal generally appears as a peak, thereby enabling the PPG signal to be used to detect peak-to-peak intervals, which can be used to calculate HR and HRV. In accordance with certain embodiments, the optical sensor 122 includes a light source that emits light of two different wavelengths that enables the optical sensor 122 to be used as a pulse oximeter, in which case the optical sensor 122 can non-invasively monitor the arterial oxygen saturation of a person wearing the device 102.

The ECG sensor 124 can be used to obtain an ECG signal from a user that is wearing the device 102 on their wrist or arm (in which case the ECG sensor 124, which is an electrode, is in contact with the user's wrist or arm), and the user touches the front facing ECG electrode 114 with a finger of their other arm. In other words, a person must complete a circuit by touching one of the ECG electrodes 114, 124 within a left portion of their body (e.g., a finger of their left hand) and touching the other one of the ECG electrodes 114, 124 with a right portion of their body (e.g., a finger of their right hand).

The skin temperature sensor 126 can be implemented, e.g., using a thermistor, and can be used to sense the temperature of a user's skin, which can be used to determine user activity and/or calories burned.

The motion sensor 128 can be an accelerometer. The accelerometer can be a three-axis accelerometer, which is also known as a three-dimensional (3D) accelerometer, but is not limited thereto. The accelerometer may provide an analog output signal representing acceleration in one or more directions. For example, the accelerometer can provide a measure of acceleration with respect to x, y and z axes. The motion sensor 128 can alternatively be a gyrometer, which provides a measure of angular velocity with respect to x, y and z axes. It is also possible that the motion sensor 128 is an inclinometer, which provides a measure of pitch, roll and yaw that correspond to rotation angles around x, y and z axes. It is also possible the device 102 includes multiple different types of motion sensors, some examples of which were just described. Depending upon the type(s) of motion sensor(s) used, such a sensor can be used to detect the posture of a portion of a person's body (e.g., a wrist or arm) on which the device 102 is being worn.

Depending upon implementation, HR and HRV can be detected based on signals obtained by the optical sensor 122 and/or the ECG sensor 124. HR and/or HRV can be automatically determined continuously, periodically or at other specified times or based on a manual user action. For example, in a free living application, HR can be determined automatically during periods of interest, such as when a significant amount of activity is detected.

Additional physiologic metrics can also be obtained using the sensors described herein. For example, respiration rate can be determined from a PPG signal obtained using the optical sensor 122 and/or from the ECG signal determined using the ECG sensor 124. For another example, blood pressure can be determined from PPG and ECG signals by determining a metric of pulse wave velocity (PWV) and converting the metric of PWV to a metric of blood pressure. More specifically, a metric of PWV can be determining by determining a time from a specific feature (e.g., an R-wave) of an obtained ECG signal to a specific feature (e.g., a maximum upward slope, a maximum peak or a dicrotic notch) of a simultaneously obtained PPG signal. An equation and or look-up-table (LUT) can then be used to convert the metric of PWV to a metric of blood pressure.

FIG. 2 depicts an exemplary block diagram of electrical components of the device 102, according to an embodiment. Referring to FIG. 2, the device 102 is shown as including a microcontroller 202 that includes a processor 204, memory 206 and a wireless interface 208. It is also possible that the memory 206 and wireless interface 208, or portions thereof, are external the microcontroller 202. The microcontroller 202 is shown as receiving signals from each of the aforementioned sensors 122, 124, 126 and 128. The device 102 is also shown as including a battery 210 that is used to power the various components of the device 102. While not specifically shown, the device 102 can also include one or more voltage regulators that are used to step-up and or step-down the voltage provided by the battery 210 to appropriate levels to power the various components of the device 102. The microcontroller 202 can also drive the digital display 108, or alternative or additional circuitry can be used to drive the display 108.

Each of the aforementioned sensors 122, 124, 126, 128 can include or have associated analog signal processing circuitry to amplify and/or filter raw signals produced by the sensors. It is also noted that analog signals produced using the aforementioned sensors 122, 124, 126, 128 can be converted to digital signals using one or more digital to analog converters (ADCs), as is known in the art. The analog or digital signals produced using these sensors can be subject time domain processing, or can be converted to the frequency domain (e.g., using a Fast Fourier Transform or Discrete Fourier Transform) and subject to frequency domain processing. Such time domain processing, frequency domain conversion and/or frequency domain processing can be performed by the processor 204, or by some other circuitry.

The device 102 is shown as including various modules, including a heart rate (HR) detector module 218, a heart rate variability (HRV) detector module 220, an activity detector module 222 and a calorie burn detector module 224. The various modules may communicate with one another. Each of these modules 218, 220, 222 and 224 can be implemented using software, firmware and/or hardware. It is also possible that some of these modules are implemented using software and/or firmware, with other modules implemented using hardware. Other variations are also possible. In accordance with a specific embodiment, each of these modules 218, 220, 222 and 224 is implemented using software code that is stored in the memory 206 and is executed by the processor 204. The memory 206 is an example of a tangible computer-readable storage apparatus or memory having computer-readable software embodied thereon for programming a processor (e.g., 204) to perform a method. For example, non-volatile memory can be used. Volatile memory such as a working memory of the processor 204 can also be used. The computer-readable storage apparatus may be non-transitory and exclude a propagating signal.

The wireless interface 206 can wirelessly communicate with a base station (e.g., 252), such as a mobile phone, a tablet computer, a PDA, a laptop computer, a desktop computer, or some other computing device that is capable of performing wireless communication. The wireless interface 206, and more generally the device 102, can communicate with a base station 252 using various different protocols and technologies, such as, but not limited to, Bluetooth™, Wi-Fi, ZigBee or ultrawideband (UWB) communication. In accordance with an embodiment, the wireless interface 206 comprises telemetry circuitry that include a radio frequency (RF) transceiver electrically connected to an antenna (not shown), e.g., by a coaxial cable or other transmission line. Such an RF transceiver can include, e.g., any well-known circuitry for transmitting and receiving RF signals via an antenna to and from an RF transceiver of a base station 252.

The HR detector module 218, which can also be referred to simply as the HR detector 218, uses signals and/or data obtained from the optical sensor 122 and/or the ECG sensor 124 to detect HR. For example, the optical sensor 122 can be used to obtain a PPG signal from which peak-to-peak intervals can be detected. For another example, the ECG sensor 124 can be used to obtain an ECG signal, from which peak-to-peak intervals, and more specifically R-R intervals, can be detected. The peak-to-peak intervals of a PPG signal or an ECG signal can also be referred to as beat-to-beat intervals, which are intervals between heart beats. Beat-to-beat intervals can be converted to HR using the equation HR=(1/beat-to-beat interval)*60. Thus, if the beat-to-beat interval=1 sec, then HR=60 beats per minute (bpm); or if the beat-to-beat interval=0.6 sec, then HR=100 bpm. The user's HR can be displayed on the digital display 108 and/or uploaded to a base station (e.g., 252) for further analysis.

The HRV detector module 220, which can also be referred to simply as the HRV detector 220, uses signals and/or data obtained from the optical sensor 122 and/or the ECG sensor 124 to detect HRV. For example, in the same manner as was explained above, beat-to-beat intervals can be determined from a PPG signal obtained using the optical sensor 122 and/or from an ECG signal obtained using the ECG sensor 124. HRV can be determined by calculating a measure of variance, such as, but not limited to, the standard deviation (SD), the root mean square of successive differences (RMSSD), or the standard deviation of successive differences (SDSD) of a plurality of consecutive beat-to-beat intervals. Alternatively, or additionally, obtained PPG and/or ECG signals can be converted from the time domain to the frequency domain, and HRV can be determined using well known frequency domain techniques. The user's HRV can be displayed on the digital display 108 and/or uploaded to a base station (e.g., 252) for further analysis.

The activity detector module 222, which can also be referred to simply as the activity detector 222, can determine a type and amount of activity of a user based on information such as, but not limited to, motion data obtained using the motion sensor 128, heart rate as determined by the HR detector 218, skin temperature as determined using the skin temperature sensor 126, and time of day. For a more specific example, the activity detector module 222 can using motion data, obtained using the motion sensor 128, to determine the number of steps that a user has taken with a specified amount of time (e.g., 24 hours), as well as to determine the distance that a user has walked and/or run within a specified amount of time. Activity metrics can be displayed on the digital display 108 and/or uploaded to a base station (e.g., 252) for further analysis.

The calorie burn detector module 224, which can also be referred to simply as the calorie burn detector 222, can determine a current calorie burn rate and an amount of calories burned over a specified amount of time based on motion data obtained using the motion sensor 128, HR as determined using the HR detector 218, and/or skin temperature as determined using the skin temperature sensor 126. A calorie burn rate and/or an amount of calories burned can be displayed on the digital display 108 and/or uploaded to a base station (e.g., 252) for further analysis.

Some people prefer not to wear anything on their wrist or arm while exercising, e.g., because they find it uncomfortable. Accordingly, rather than strapping the device 102 on their wrist or arm, some people may hold the device 102 in one hand while exercising, which is inconvenient and/or cumbersome. Additionally, where the person needs both of their hands to perform an exercise, they may end up putting down the device 102, during which period of time the device will not perform many of its intended functions. Other people may choose to place the device 102 in a pocket, where it is not viewable and is often uncomfortable. Depending upon what features are provided by the device 102, certain features may not function unless the device 102 is in contact with a user's skin, and thus, some features may not function when the device 102 is placed in a pocket or set down. For example, in order for the optical sensor 122 and/or the ECG sensor 124 to obtain measures of HR, HRV, blood oxygen concentration and/or blood pressure, these sensors must contact a person's skin. Other features of the device 102 may function correctly, so long as the device as attached to a person's clothes. For example, at least some activity metrics that are determined using the motion sensor 128 can be determined so long as the device 102 is attached to a person's clothes.

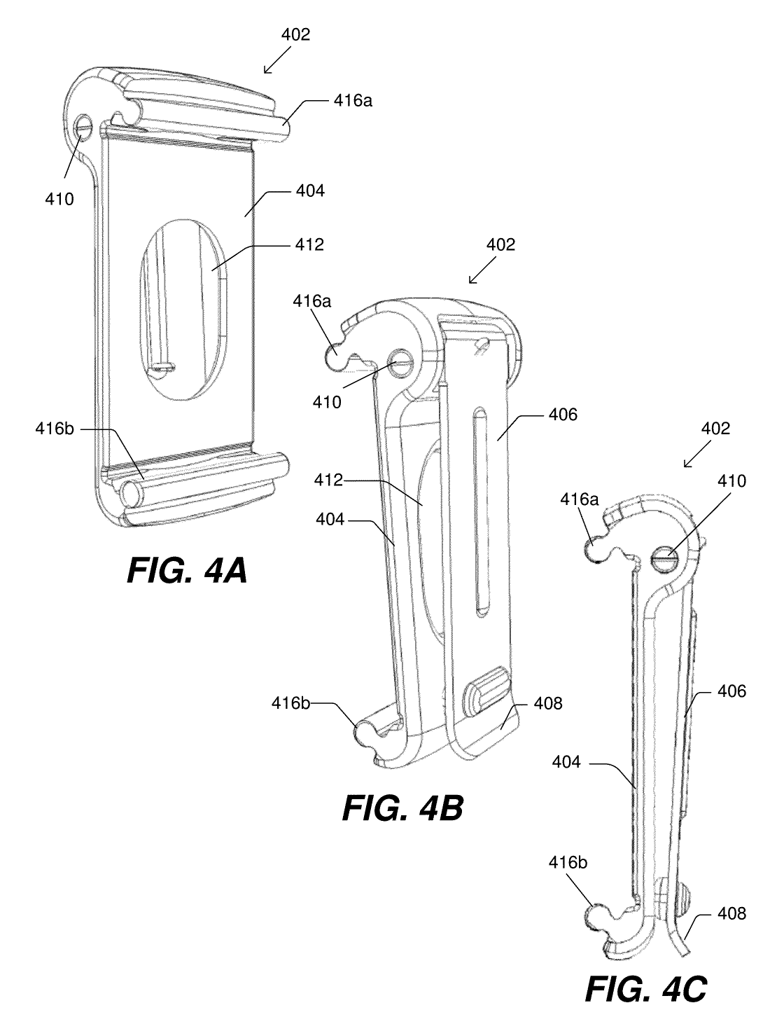

Specific embodiments of the present invention, which are describe below with reference to FIGS. 4A-9, relate an adaptor that can be selectively mated with the device 102 to enable the device to be clipped to a person's finger or clothes. However, prior to mating the adaptor with the device 102, the first and second portions of the band 106a, 106b should first be detached from the device 102. In an embodiment, the first and second portions of the band 106a, 106b can be detached from the device 102 by sliding connectors of the first and second portions of the band 106a, 106b out of slots of the device 102. Alternatively, the first and second portions of the band 106a, 106b can be detached from the device 102 by unsnapping connectors of the first and second portions of the band 106a, 106b from slots of the device 102.

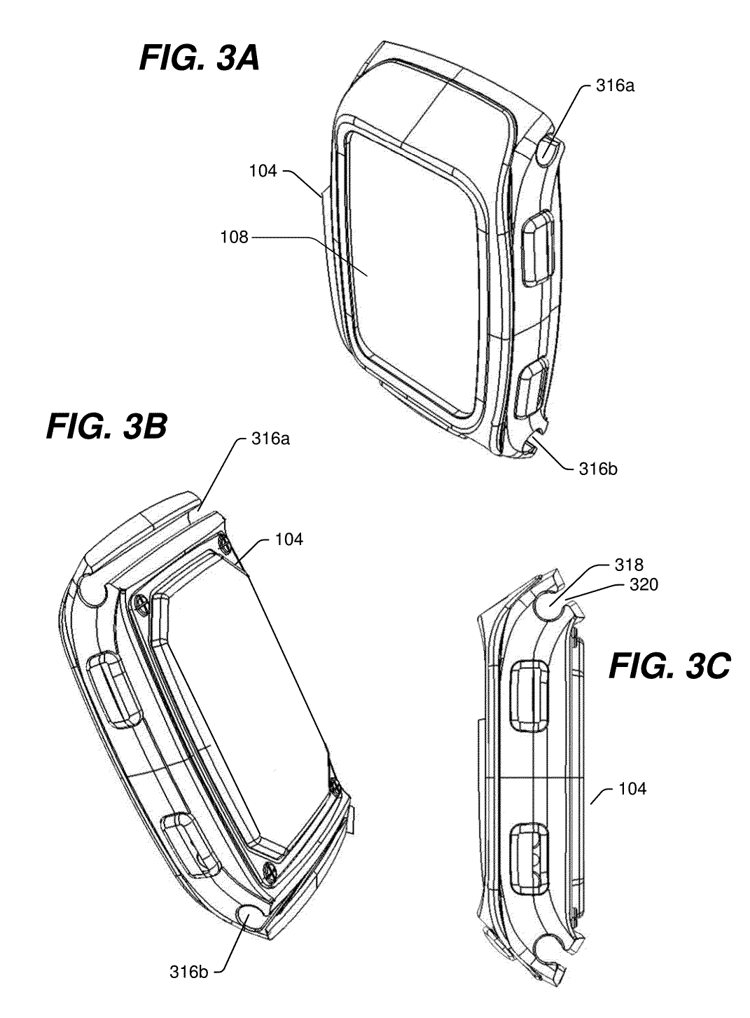

FIGS. 3A, 3B and 3C respectively show front perspective, rear perspective and side views of the device 102 after the first and second portions of the band 106a, 106b have been detached from the device 102. Referring to FIGS. 3A, 3B and 3C, the device 102 is shown as including first and second slots 316a, 316b located at first and second opposing ends of the device 102. The slots can individually be referenced as a slot 316, and can collectively be references as the slots 316. The slots 316 extend in a straight line across a width of the device 102. More specifically, the slots 316 are arranged orthogonal to the longitudinal axis of the device 102. As shown in FIG. 3C, each slot 316 includes a relatively enlarged portion 318 a relatively narrow portion 320. This configuration of the slots 316 allows connectors of the first and second portions of the band 106a, 106b, which have a bulbous profile, to be slid into or snapped into the slots 316. This configuration of slots 316 also allows connectors of the first and second portions of the band 106a, 106b to be slid out of or snapped out of the slots 316. While not specifically shown in FIG. 3B, the backside of the housing 104 can include one or more sensors intended to contact a person's skin, examples of which were described above with reference to FIGS. 1B and 2.

FIGS. 4A, 4B and 4C respectively show front perspective, rear perspective and side views of an adaptor 402 that can be selectively mated with the device 102 to enable the device to be clipped to a person's finger or clothes. The adaptor 402 includes a base 404 having first and second ends with a longitudinal axis of the base 404 extending between the first and second ends of the base. Additionally, the adaptor 402 includes a lever 406 having first and second ends with a longitudinal axis of the lever 406 extending between the first and second ends of the lever 406 and being parallel to the longitudinal axis of the base 404. A hinge 410 connects the first end of the lever 406 to the first end of the base 404 and biases the second end of the lever 406 towards the second end of the base 402. A spring (e.g., a plate spring or a coil spring, not shown) can provide the biasing of the second end of the lever 406 towards the second end of the base 402. The second end of the lever 406 includes a lip 408 that bends or extends in a direction away from the base 404. This lip 408 enables the lever 406 to be more easily be slid over an article of clothes when the adaptor 402 is being used to clip the device 102 to an article of clothes. The lip 408 also improves comfort when the adaptor 402 is used to clip the device 102 to a person's finger.

First and second connector 416a, 416b extend from the base 404 and are configured to fit within the slots 316 of the device 102, to thereby mate the adaptor 402 to the device 102. The first and second connector 416a, 416b can be referenced individually as a connector 416, or collectively as the connectors 416. As can be appreciated from FIG. 4A, the connectors 416 are arranged orthogonal to the longitudinal axis of the base 404. In accordance with the embodiment shown, each of the connectors 416 has a bulbous profile.

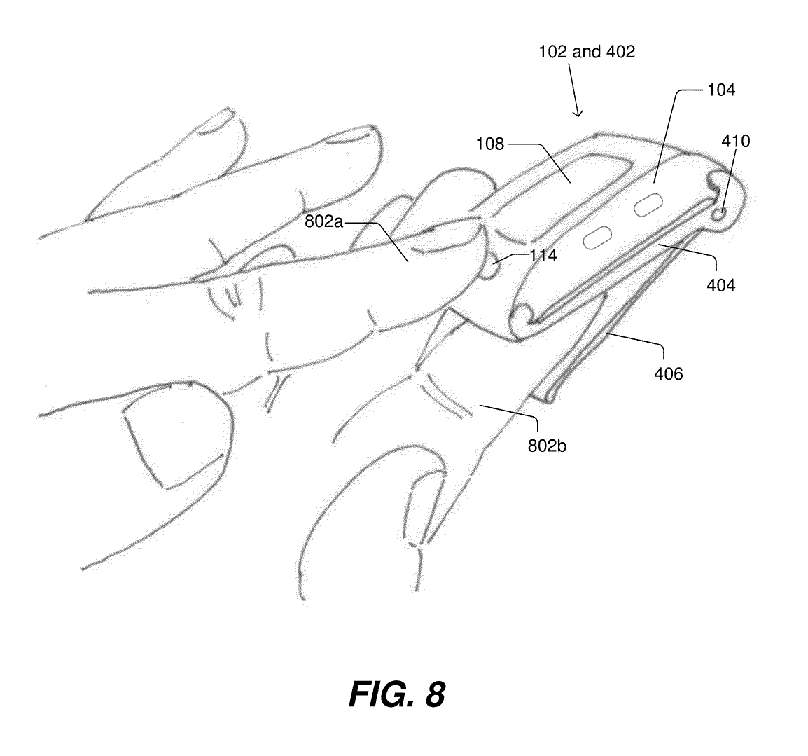

The base 404 also includes an opening 412 that is sized and positioned to enable a distal portion of a person's finger to extend through the opening 412 and contact the device 102 when the adaptor 402 is mated to the device 102 and the device is clipped to a person's finger, e.g., as shown in FIG. 8 discussed below. The length of the opening 412 is preferably at least 0.5 inches, and preferably 0.75 inches or longer. The width of the opening 412 is preferably at least 0.25 inches, and preferably 0.5 inches or wider.

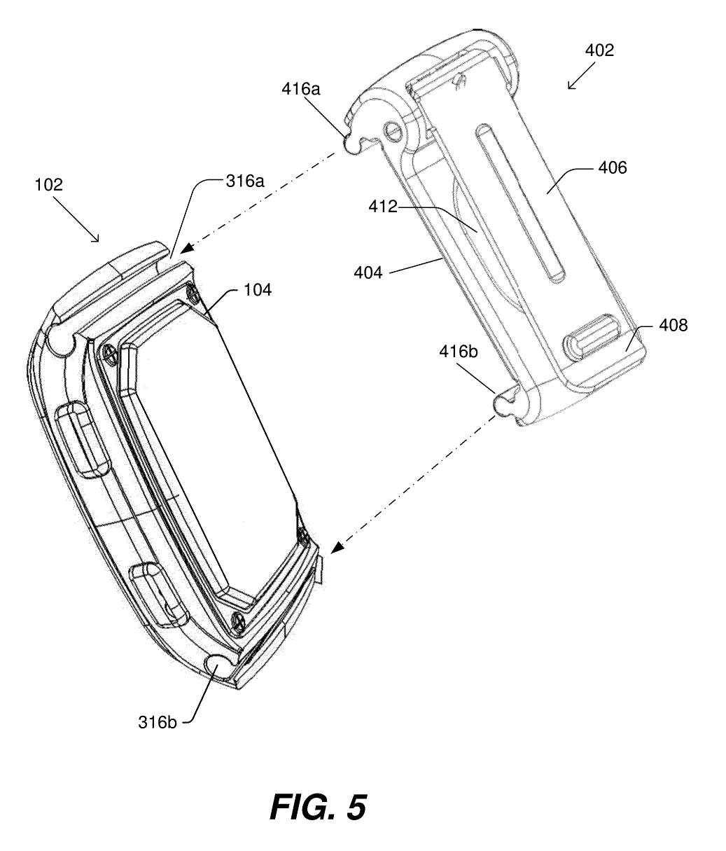

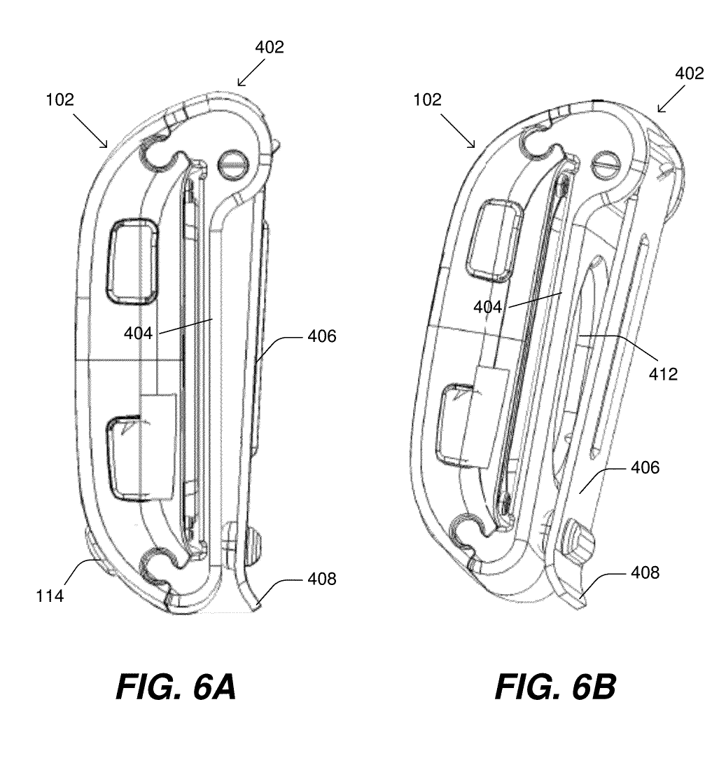

FIG. 5 illustrates how the connectors 416 of the adapter 402 can be slid into the slots 316 of the device 102. Alternatively, or additionally, the connectors 416 of the adaptor 402 can be snapped into the slots 316 of the device 102. When the adaptor 402 is mated with the device 102, the lever 406 of the adaptor 402 can be used to clip that device 102 onto a person's belt, pants packet, shirt pocket, shirt neck, or other article of clothing or portion thereof. Alternatively, when the adaptor 402 is mated with the device 102, the lever 406 of the adaptor 402 can be used to clip that device 102 to a person's finger in a similar manner that a finger sensor probe of a pulse oximeter used by a hospital clips to a person's finger. As mentioned above, in the discussion of FIGS. 4A, 4B and 4C, the base 402 includes the opening 412 that enables a distal portion of a person's finger to extend through the opening 412 and contact the device 102 when the adaptor 402 is mated to the device 102 and used to clip the device 102 to a person's finger. This is beneficial where the backside of the housing 104 of the device 102 includes one or more sensors that need to contact a person's skin for the sensor(s) to function correctly. Examples of such sensors include the optical sensor 122, the ECG sensor 124 and the skin temperature sensor 126, which were discussed above with reference to FIGS. 1B and 2. The device 102 can include all three of these sensors, or just one or two of the sensors. The device may also include addition or alternative types of sensors that are intended to contact a persons skin. FIGS. 6A and 6B are respectively side and perspective views of the adaptor 402 mated with the device 102.

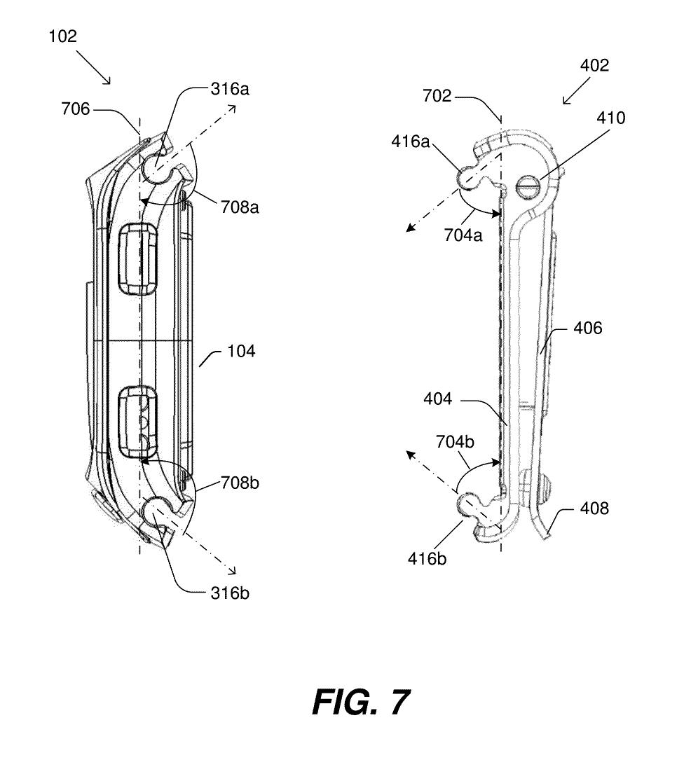

FIG. 7, which illustrates side views of device 102 and the adaptor 402 detached from one another, will now be used to describe some additional details about the connectors 416 of the adaptor 402. Referring to the adaptor 402 at the right side in FIG. 7, the connectors 416a, 416b are shown as extending from the base 404 in directions that converge toward one another at acute angles 704a, 704b relative to the longitudinal axis 702 of the base 404. Referring to the device 102 at the left side in FIG. 7, the slots 316 of the device 102 are shown as diverging away from one another at obtuse angles 708a, 708b relative to the longitudinal axis 706 of the device 102. In accordance with an embodiment, the acute angle 704 at which one of the connectors 416 extends from the base 404 of the adaptor 402 is supplementary with the obtuse angle 708 of the slot 316 of the device 102 with which the connector 416 is configured to mate. In other words, the angles 704 and 708 add up to 180 degrees. In accordance with an embodiment, each of the angles 704a, 704b is approximately 45 degrees, and each of the angle 708a, 708b is approximately 135 degrees. This configuration provides for a secure mating between the adaptor 402 and the device 102.

FIG. 8 illustrates the device 102 with the adapter 402 mated therewith clipped to a finger 802a of a person's right hand, while a finger 802b of the person's left hand is touching the outwardly facing ECG sensor 114. While it cannot be seen in FIG. 8, a distal portion of the finger 802a on the person's right hand extends through the opening 412 in the base 402 of the adaptor 402 and touches the ECG sensor 124 that is on the backside of the housing 104. This completes a circuit through a portion of the person's body that includes their heart, thereby enabling an ECG signal to be obtained. As mentioned above, such an ECG signal can be used to measure physiologic metrics, such as, but not limited to, HR and HRV. If the backside of the housing of the device 102 includes the optical sensor 122, then the distal portion of the finger 702 on the person's left hand that extends through the opening 412 in the lever 406 of the adaptor 402 will contact the optical sensor 122, which will enable a PPG signal to be obtained. As mentioned above, such a PPG signal can be used to measure physiologic metrics, such as, but not limited to, HR and HRV. The optical sensor 122 can also enable the device 102 to function as a pulse oximeter and obtain measures of oxygen saturation. If the device 102 includes both an optical sensor and an ECG sensor, then the device can determine a metric of PWV, which as mentioned above, can be used by the device 102 to determine the person's blood pressure. If the backside of the housing of the device 102 includes the skin temperature sensor 126, then the distal portion of the finger 702 on the person's left hand that extends through the opening 412 in the base 406 of the adaptor 402 will contact the skin temperature sensor 126 so that the device can determine the temperature of the person's skin. Such information can be used, e.g., to monitor activity and/or determine a calorie burn rate.

Embodiments of the present invention are also directed to systems that include a device, such as the activity monitor device 102, a detachable band (e.g., 106) that enables the device to be strapped to a persons' wrist, and an adaptor (e.g., 402) that enables the device to be clipped to a person's finger or clothes when the band is detached from the device. Such a system may be sold in a package that include the device, the band and the adaptor, to thereby enable a person to customize whether they want to attach the band or the adaptor to the device. In other words, the band and the adaptor can be interchangeable, so that at any given time one of the band and the adaptor can be attached to the device.

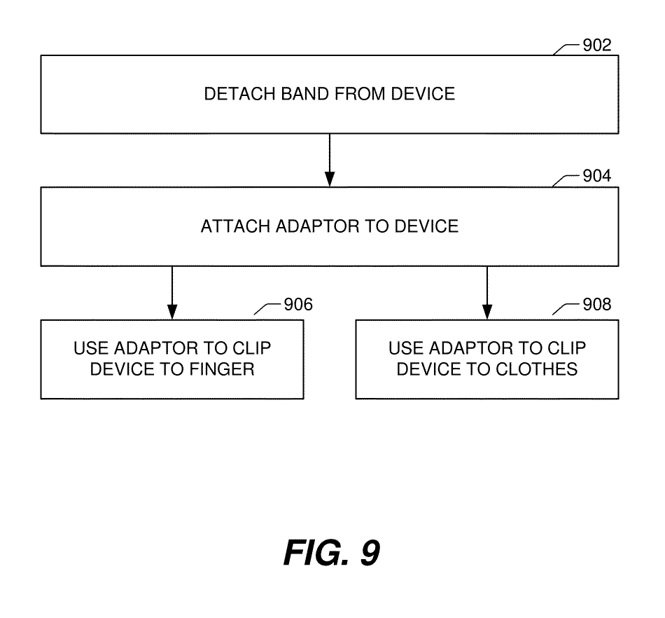

FIG. 9 is a high level flow diagram that is used to describe various methods according to embodiments of the present invention. Some such methods are for use with a device (e.g., 102) having a band (e.g., 106) that can be used to strap the device to a person's wrist. Even more specifically, such methods enable such a device to be clipped to a person's finger or clothes, instead of being strapped to a person's wrist.

Referring to FIG. 9, at step 902, the band is detached from the device. Step 902 can include detaching a first portion of the band from a first slot of the device and detaching a second portion of the band from a second slot of the device. More specifically, step 902 can include sliding a connector of the first portion of the band out of the first slot of the device and sliding a connector of the second portion of the band out of the second slot of the device. Alternatively, step 902 can include unsnapping a connector of the first portion of the band from the first slot of the device and unsnapping a connector of the second portion of the band from the second slot of the device.

At step 904, an adaptor is attached to the device, wherein the adaptor is configured to be clipped to a person's finger or clothes. An example of such an adaptor is the adaptor 402 that was described above with reference to FIGS. 4A-8. Step 904 can include mating first and second connectors of the adaptor respectively with first and second slots of the device to thereby attach the adaptor to the device. More specifically, step 904 can include sliding the first and second connectors of the adaptor respectively into the first and second slots of the device to thereby attach the adaptor to the device. Alternatively, step 904 can include snapping the first and second connectors of the adaptor respectively into the first and second slots of the device to thereby attach the adaptor to the device.

At step 906, the device is clipped to a finger. At step 908, the device is clipped to an article of clothing. In certain embodiments, only one of steps 906 and 908 are performed. Steps 906 and 908 can be performed one after the other, in either order. The method can also include detaching the adaptor from the device, and attaching the band to the device so the device can once again be worn on a user's wrist.

As the terms are used herein, the term mating and attaching are used interchangeably. Similarly, the terms mated and attached are used interchangeably. Additional, the term person and user are used interchangeably.

In the above description, the device 102 was generally described as being an activity monitor device that can determine and track over time various metrics, such as, but not limited to, HR, HRV, calories burned, steps taken, and/or distance walked and/or ran. The device 102 was also described as being able to determine and track over time oxygen saturation and blood pressure. The device 102 can also determine and track additional and/or alternative metrics than those specifically described herein. Further, if the device 102 includes a wireless interface (e.g., 206), the device 102 can receive alerts from a base station (e.g., 252). Exemplary types of alerts were discussed above in the discussion of FIG. 2. The device 102 may also be able to upload data to and download data from a base station.

The foregoing detailed description of the technology herein has been presented for purposes of illustration and description. It is not intended to be exhaustive or to limit the technology to the precise form disclosed. Many modifications and variations are possible in light of the above teaching. The described embodiments were chosen to best explain the principles of the technology and its practical application to thereby enable others skilled in the art to best utilize the technology in various embodiments and with various modifications as are suited to the particular use contemplated. It is intended that the scope of the technology be defined by the claims appended hereto. While various embodiments have been described above, it should be understood that they have been presented by way of example, and not limitation. It will be apparent to persons skilled in the relevant art that various changes in form and detail can be made therein without departing from the spirit and scope of the invention. The breadth and scope of the present invention should not be limited by any of the above-described exemplary embodiments, but should be defined only in accordance with the following claims and their equivalents.

What is claimed is:

1. An adaptor configured to be selectively mated with a device to enable the device to be clipped to a person's finger or clothes, the adaptor comprising:
a base having first and second ends with a longitudinal axis of the base extending between the first and second ends of the base;
a lever having first and second ends with a longitudinal axis of the lever extending between the first and second ends of the lever and being parallel to the longitudinal axis of the base;
a hinge that connects the first end of the lever to the first end of the base and biases the second end of the lever towards the second end of the base; and
at least one connector extending from the base and configured to mate with a portion of the device to thereby selectively mate the adaptor with the device;
wherein the base includes an opening sized and positioned to enable a distal portion of a person's finger to extend through the opening and contact the device when the adaptor is mated to the device and the device is clipped to a person's finger.

2. The adaptor of claim 1, wherein the device includes first and second slots, and wherein:
the at least one connector comprises first and second connectors extending from the base;
wherein the first and second connectors are configured to mate with first and second slots of the device to thereby selectively mate the adaptor with the device.

3. The adaptor of claim 2, wherein the first and second connectors are arranged orthogonal to the longitudinal axis of the base.

4. The adaptor of claim 2, wherein the first and second connectors have a bulbous profile.

5. The adaptor of claim 2, wherein the first and second connectors extend from the base in directions that converge toward one another at acute angles relative to the longitudinal axis of the base.

6. The adaptor of claim 2, wherein the first and second connectors are configured to slide into the first and second slots of the device to thereby selectively mate the adaptor with the device.

7. The adaptor of claim 2, wherein the first and second connectors are configured to snap into the first and second slots of the device to thereby selectively mate the adaptor with the device.

8. A method for use with a device having a band that can be used to strap the device to a person's wrist, the method for enabling the device to be clipped to a person's finger or clothes instead of being strapped to a person's wrist, the method comprising:
(a) detaching the band from the device; and
(b) attaching an adaptor to the device, wherein the adaptor is configured to be clipped to a person's finger or clothes.

9. The method of claim 8, further comprising:
(c) clipping the device, with the adaptor attached thereto, to a finger.

10. The method of claim 8, further comprising:
(d) clipping the device, with the adaptor attached thereto, to an article of clothing.

11. The method of claim 8, wherein the device includes first and second slots that are respectively mateable with first and second portions of the band that can be used to strap the device to a persons' wrist, wherein:
step (a) includes detaching the first portion of the band from the first slot of the device and detaching the second portion of the band from the second slot of the device; and
step (b) includes mating first and second connectors of the adaptor respectively with the first and second slots of the device to thereby attach the adaptor to the device.

12. The method of claim 11, wherein:
step (a) includes sliding a connector of the first portion of the band out of the first slot of the device and sliding a connector of the second portion of the band out of the second slot of the device; and
step (b) includes sliding the first and second connectors of the adaptor respectively into the first and second slots of the device to thereby attach the adaptor to the device.

13. The method of claim 11, wherein:
step (a) includes unsnapping a connector of the first portion of the band from the first slot of the device and unsnapping a connector of the second portion of the band from the second slot of the device; and
step (b) includes snapping the first and second connectors of the adaptor respectively into the first and second slots of the device to thereby attach the adaptor to the device.

14. A system, comprising:
an activity monitor device including a display;
an adaptor configured to be selectively mated with the device;
wherein when mated to the device the adaptor enables the device to be clipped to a person's finger or clothes;
wherein the device includes one or more sensors selected from the group consisting of an ECG sensor, an optical sensor and a skin temperature sensor; and
wherein the adaptor includes
a base;
a lever;
a hinge that connects a first end of the lever to a first end of the base and biases a second end of the lever towards a second end of the base; and
wherein the base includes an opening sized and positioned to enable a distal portion of a person's finger to extend through the opening and contact the device when the adaptor is mated to the device and the device is clipped to a person's finger.

15. The system of claim 14, further comprising:
a band configured to be selectively mated to the device, wherein when mated to the device the band enables the device to be strapped to a person's wrist;
wherein at any given time, either the adaptor or the band can be mated to the device, thereby enabling a person to choose whether to strap the device to their wrist, clip the device to one of their fingers, or clip the device to an article of clothing.

16. The system of claim 15, wherein:
the device is configured to determine one or more metrics selected from the group consisting of heart rate, heart rate variability, calories burned, steps taken, or distance walked and/or ran;
at least one of the one or more sensors is positioned on the device such that the at least one of the one or more sensors contacts a person's skin when the device is worn on a person's wrist using the band, as well as when the device is worn on a person's finger using the adaptor.

17. The system of claim 14, wherein:
the device includes first and second slots;
the adaptor includes first and second connectors extending from the base;
wherein the first and second connectors of the adaptor are configured to mate with the first and second slots of the device to thereby selectively mate the adaptor with the device.

18. The system of claim 17, wherein the first and second connectors of the adaptor are arranged orthogonal to the longitudinal axis of the base of the adaptor.

19. The system of claim 17, wherein the first and second connectors have a bulbous profile.

20. The system of claim 17, wherein:
the first and second slots of the device diverge away from one another at obtuse angles relative to a longitudinal axis of the device;
the first and second connectors of the adaptor extend from the base in directions that converge toward one another at acute angles relative to a longitudinal axis of the base; and
a said acute angle and a said obtuse angle are supplementary angles.
